(19) United States
(12) Patent Application Publication
(10) Pub. No.: US 2015/0359652 A1
(43) Pub. Date: Dec. 17, 2015

(54) TOE CORRECTION DEVICE

(71) Applicant: Mi-nam LEE, (US)

(72) Inventor: Mi-nam LEE, Gyeongsan-si, Gyeongsangbuk-do (KR)

(21) Appl. No.: 14/764,181

(22) PCT Filed: Feb. 3, 2014
(86) PCT No.: PCT/KR2014/000904

§ 371 Date: Jul. 29, 2015

(30) Foreign Application Priority Data
Jan. 30, 2013 (KR) 10-2013-0010567

(51) Int. Cl. A61F 5/01

(52) U.S. Cl. CPC A61F 5/019

(57) ABSTRACT

The present invention relates to a toe correction device including an elastic body readily worn on the foot while preventing inadvertent removal thereof, a toe insertion opening formed in the front of the body for inserting each of the toes so as to expose the front part of the toes, and a thick resilient correction part arranged between adjacent toe insertion openings so as to correct the toes while the user is freely moving, and to massage the toes during walking, thus assisting the blood circulation.

### TECHNICAL FIELD

The present invention relates to a toe correction device, and more particularly to a toe correction device that can be easily worn on a foot to stretch the foot, having been squeezed in a shoe all day long, in order to correct toe deformation, such as hallux valgus.

### BACKGROUND ART

Modern people, who are very active outside the home, wear shoes all day long. For this reason, toes are maintained close to each other without sufficient space in which the toes can move freely. As a result, modern people may contract diseases, such as athlete's foot. In particular, modern women frequently wear narrow shoes, such as high-heeled shoes, with the result that the toes may be bent, and, in the worst case, hallux valgus may occur. In addition, in recent years, even shoes for children have followed those for adults in terms of shape and style. As a result, even children may suffer from deformation of the feet.

Hallux valgus is a condition in which the big toe is bent toward the second toe, and a joint protrudes inward. The protruding joint is stimulated while standing or walking, with the result that the joint becomes red. In addition, corns occur on the big toe, and the big toe becomes inflamed and painful. Because the big toe, which is supposed to support the foot and to provide propulsive force to the foot during walking, malfunctions, pain is also experienced in other portions of the foot. In the worst case, the foot becomes deformed, and pain is felt even in the waist.

One toe correction device which has been widely used to correct such deformation of the toes in recent years is configured such that correction members made of a material, such as silicon, which exhibits elasticity, are inserted between the respective toes. An example of such a toe correction device is disclosed in Korean Utility Model Registration No. 20-0390011. However, the disclosed toe correction device does not include a means for fixing the correction members to prevent the correction members from becoming separated from the foot. For this reason, it is necessary to put a sock or a stocking on the foot after inserting the correction members between the respective toes, which is inconvenient. Furthermore, the correction members may become dislodged from their positions even while the sock or the stocking is worn on the foot. As a result, wear comfort is deteriorated, and the desired correction effect is not achieved.

Meanwhile, the applicant of the present application filed a utility model application entitled ‘anti-slip foot-wearable device’ for preventing slippage and additionally the above-described correction of the toes, which has been assigned Korean Utility Model Registration No. 20-0459932. The prevent invention is characterized by correction of the toes.

### DISCLOSURE

### Technical Problem

The present invention has been made in view of the above problems, and it is an object of the present invention to provide a toe correction device that is capable of correcting and stretching toes, thereby preventing deformation of a foot and preventing pain in the waist due to the deformation of the foot, the toe correction device being configured to have a structure in which the foot is inserted through an elastic body such that the body can be easily worn on the foot and cannot be separated from the foot, toe insertion holes corresponding to the respective toes are provided at the front of the body, and elastic correction members, each of which has a predetermined thickness, are provided between the toe insertion holes, whereby the toes are smoothly stretched through shrinkage and relaxation of the foot, while the joints of the toes are frequently used, i.e. when walking.

It is another object of the present invention to provide a toe correction device that is capable of correcting toes while allowing the toes to move freely as if in a barefoot state.

### Technical Solution

In accordance with an aspect of the present invention, the above and other objects can be accomplished by the provision of a toe correction device including an elastic body formed such that the fore part of a foot, including toes, can be inserted through the elastic body, toe insertion holes formed in the front of the body such that the toes can be inserted through the respective toe insertion holes in a state in which the toes are partially exposed, and elastic correction members, each having a predetermined thickness, provided between the respective toe insertion holes.

The toe correction device may be integrally manufactured by injection molding using a resin, such as silicon, which exhibits elasticity. Alternatively, the body may be formed of cloth, and the correction members may be fixed at appropriate positions through sewing.

In addition, an elastic band may be provided at one end of the body to prevent the toe correction device from being separated from the foot while the toe correction device is being worn on the foot.

The body may be provided at the bottom thereof with anti-slip members. The anti-slip members may be widely formed only at portions of the bottom of the body corresponding to points at which force is applied during walking, i.e. a bone portion from which the big toe is formed and a bone portion from which the little toe is formed. Alternatively, a single anti-slip member may be uniformly formed over the entire bottom of the body in a dot pattern. The anti-slip members may be formed such that the anti-slip members are cushioned.

### ADVANTAGEOUS EFFECTS

When the toe correction device with the above-stated construction according to the present invention is worn, the correction members, each of which has elasticity and a predetermined thickness, are located between the respective toes for separating the toes from each other, whereby it is possible to correct the toes and to prevent deformation of the foot.

In addition, the toes are stretched through shrinkage and relaxation of the foot, while the joints of the toes are frequently used, due to the toe insertion holes and the correction members, each of which has elasticity and a predetermined thickness, while a user walks, whereby it is possible to promote blood circulation and thus to reduce fatigue of the foot.

Furthermore, the body of the toe correction device is formed in the shape of a band, and an elastic band is provided at the front and/or the rear of the body, whereby it is possible to conveniently wear the toe correction device without the toe correction device being separated from the foot even when only the toe correction device is worn at ordinary times without any auxiliary means. Consequently, it is possible to wear the toe correction device indoors or to intermittently wear the toe correction device indoors while resting, thereby achieving excellent stretching and foot correction effects.

In addition, the body is provided at the bottom thereof with anti-slip members for functioning as a cushion that absorbs shocks while preventing slippage of the foot.

Furthermore, the toe insertion holes are formed such that the toes can be inserted through the respective toe insertion holes in a state in which the toes are partially exposed. As a result, the toes can move freely, whereby the toe correction device is conveniently used.

In addition, the toe correction device is made of an elastic material. Consequently, the toe correction device compresses the foot when the foot is raised, and the toe correction device expands according to the shape of the bottom of the foot when the foot is put onto the ground. As a result, the foot is stimulated, whereby blood circulation is promoted.

### DESCRIPTION OF DRAWINGS

FIG. 1 is a sectional view showing a toe correction device according to an embodiment of the present invention;

FIG. 2 is a sectional view showing a toe correction device according to another embodiment of the present invention; and

FIG. 3 is a bottom view showing a state in which the toe correction device according to the present invention is worn.

### BEST MODE

Hereinafter, an embodiment of the present invention will be described with reference to the accompanying drawings.

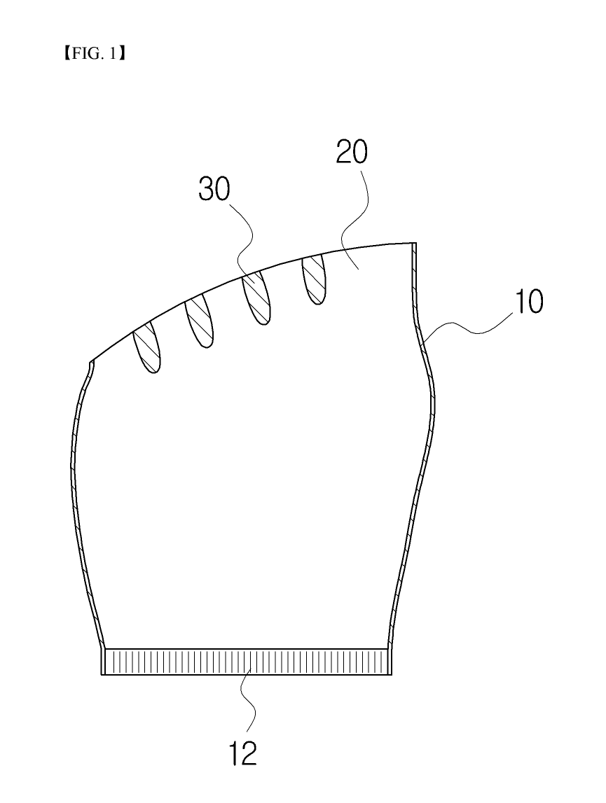

As shown in FIGS. 1 to 3, a toe correction device according to the present invention includes an elastic body 10 formed in the shape of a band to enable the insertion of the fore part of a foot, including toes, through the elastic body 10, toe insertion holes 20 formed in the front of the body 10 such that the toes can be inserted through the respective toe insertion holes 20, and elastic correction members 30, each having a predetermined thickness, provided between the respective toe insertion holes 20.

As shown in FIG. 1, the toe correction device 30 may be manufactured by injection molding using a resin, such as silicon, which exhibits elasticity, such that the toe insertion holes 20 and the correction members 30 can be formed integrally with the body 10.

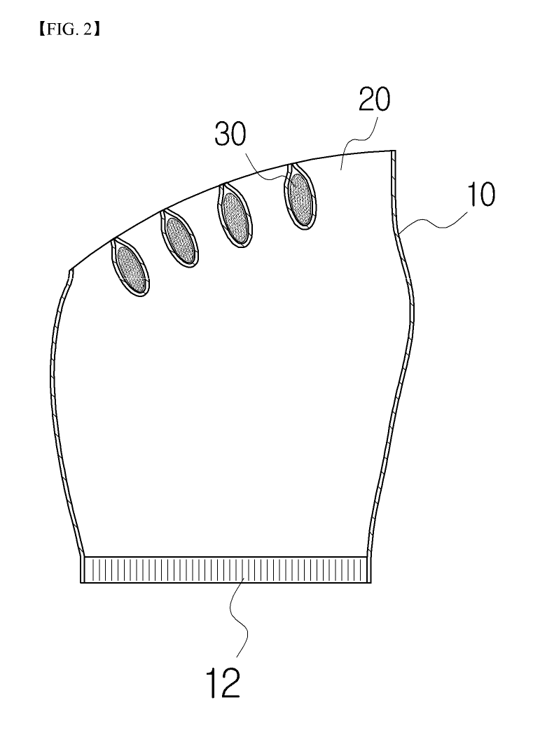

In addition, as shown in FIG. 2, the toe correction device 30 may be manufactured by forming the body 10 of cloth such that the body 10 exhibits elasticity. At this time, the toe insertion holes 20 may be formed integrally with the body 10. Alternatively, the toe insertion holes 20 may be formed separately from the body 10 through additional sewing. The correction members 30 may be inserted between the respective toe insertion holes 20, and then opposite ends of the respective toe insertion holes 20 may be sewed so as to fix the correction members 30.

The toe insertion holes 20 may be different in thickness from the correction members 30 such that the toes can be stretched, and blood circulation can be promoted, through shrinkage and relaxation of the foot during walking

The cloth may be manufactured using yarn, such as synthetic fibers, which does not absorb moisture. An example of the synthetic fibers may include polyester or polyurethane. It is not easy to dye polyester. For this reason, polyurethane is preferably used alone, or polyurethane is preferably used together with polyester. Other synthetic fibers may also be used.

Meanwhile, an elastic band 12 may be provided at one end of the body 10 of the toe correction device, i.e. the end of one side of the body 10, to prevent the toe correction device from being separated from the foot while the toe correction device is being worn on the foot. The elastic band 12 may be formed integrally with the body 10 by injection molding. Alternatively, when the body 10 is formed of the cloth through weaving or knitting, the elastic band 12 may be formed integrally with the body 10 by weaving or knitting.

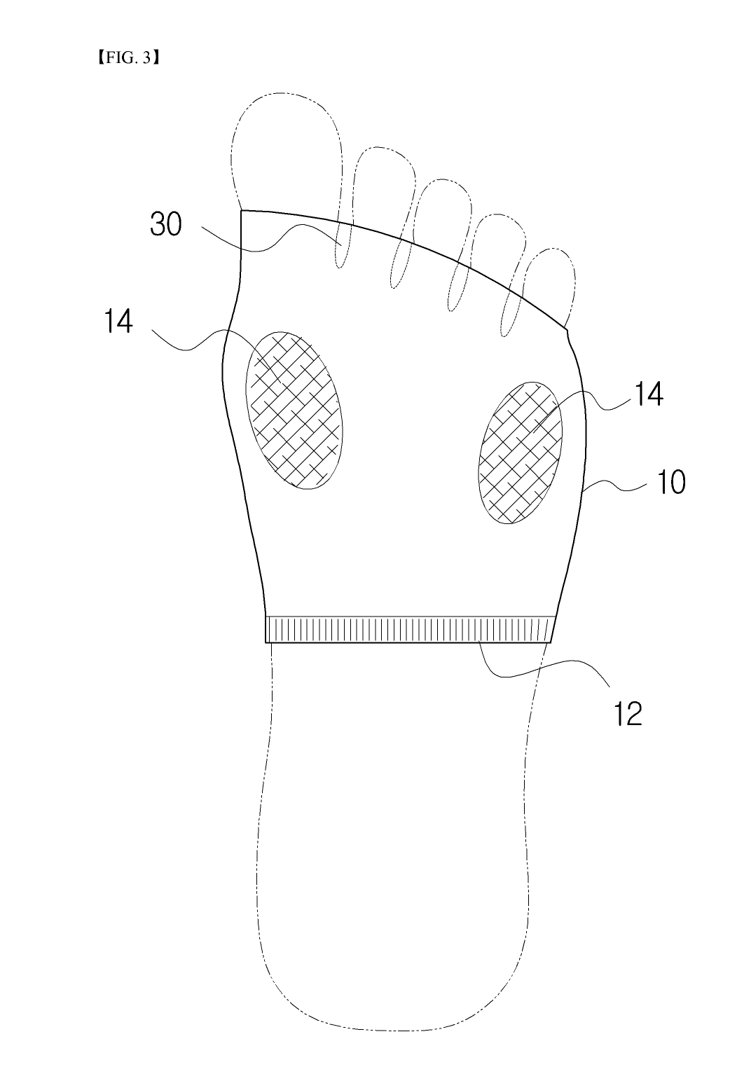

In addition, as shown in FIG. 3, the body 10 of the toe correction device may be provided at the bottom thereof with anti-slip members 14. A single anti-slip member 14 may be uniformly formed over the entire bottom of the body 10 in a dot pattern. Alternatively, two anti-slip members 14 may be widely formed only at portions of the bottom of the body 10 that correspond to the big toe and the little toe. The anti-slip members 14 may be formed by additionally applying a resin to the bottom of the body 10 such that the body 10 is cushioned. Alternatively, the anti-slip members 14 may be formed integrally during injection molding of the toe correction device.

Furthermore, plantar pressure massage protrusions (not shown) may be formed at the inner surface of the body 10.

According to the present invention as described above, as shown in FIG. 3, slippage is prevented, and the toe correction device is prevented from being separated from the foot due to the elastic band 12 while the toe correction device is being worn on the foot. In addition, the toes are separated from each other by the correction members 30, whereby it is possible to correct deformation of the toes, such as hallux valgus. Furthermore, the foot is stimulated by the anti-slip members 14 and the plantar pressure massage protrusions 16, and the body 10 is elastic, whereby it is possible to promote blood circulation.

1. A toe correction device comprising:
an elastic body (10), through which a foot is inserted such that the body is worn on the foot;
toe insertion holes (20) formed in a front of the body (10) such that toes are inserted through the respective toe insertion holes in a state in which the toes are partially exposed; and
elastic correction members, each having a predetermined thickness, provided between the respective toe insertion holes, wherein
the toe insertion holes (20) and the correction members (30) are formed integrally with the body (10) by injection molding using a resin that exhibits elasticity.

2. The toe correction device according to claim 1, wherein the body (10) is formed in a shape of a band to enable insertion of a fore part of the foot, comprising the toes, through the elastic body, and exhibits elasticity.

3. The toe correction device according to claim 2, wherein the body (10) is provided at one end thereof with an elastic band (12).

4. The toe correction device according to claim 1, wherein the body (10) of the toe correction device is provided at a bottom thereof with an anti-slip member (14).
